(19) United States
(12) Patent Application Publication
(10) Pub. No.: US 2022/0246055 A1
(43) Pub. Date: Aug. 4, 2022

(54) DEVICE AND METHOD FOR ANALYSING THE BEHAVIOUR OF A SUBJECT

(71) Applicant: THALES, Courbevcie (FR)

(72) Inventors: Serge COUVET, Lège-Cap-Ferret (FR); Pierre COUSIN, Maisons-Laffitte (FR); Yannick JAMES, Osny (FR); Pascal PEYRONNET, Paris (FR); Quentin MICHAL, Courbevoie (FR); Pierre-Emmanuel GUILLOU, Cergy (FR)

(21) Appl. No.: 17/625,755

(22) PCT Filed: Jun. 25, 2020
(86) PCT No.: PCT/EP2020/067794

§ 371 Date: Jan. 9, 2022

(30) Foreign Application Priority Data
Jul. 11, 2019 (FR) FR1907793

(51) Int. Cl. G09B 9/16; A61B 5/00

(52) U.S. Cl. CPC G09B 9/16; A61B 5/72; A61B 5/0004

(57) ABSTRACT

A device and a method are provided for analyzing the behavior of a subject moving in an operational environment during a mission, including: synchronized acquisition of a plurality of raw data relating to the subject, to the operational environment and to the mission; on-the-fly cross-processing of the acquired raw data; and real-time generation of processed data from the cross-processing operations, providing instantaneous information about the state and the behavior of the subject; the device is characterized in that all or some of the steps may be implemented so as to activate various operating modes of analyzing and monitoring the behavior of the subject.

### TECHNICAL FIELD

The invention relates to the field of behavior analysis, and proposes a device and a method for analyzing the behavior of a subject or of members of a crew, notably a crew on board an aeronautical platform.

### PRIOR ART

In many industrial fields and in particular in the aeronautical field, a new need is emerging to be able to measure and analyze the state and the behavior of a subject or of a crew during its mission, not for the purpose of research and experimentation on human behavior, but for the purpose of improving the implementation of the operational mission
improving safety, adapting to the operational situation—and that is also able to be used for education and training phases—selection, measurement of learning, tailoring of the exercise to crew responses and behavior.

This need had not been addressed until now with the same aims; it mainly involved taking measurements on the subjects (crew) and then, in a screening phase following the acquisition of the measurements using physiological sensors, analyzing states and behaviors, the approach being more focused on scientific work than on immediate operational use. The solutions in these existing approaches are essentially characterized by:
outside-real-time processing of the behavior analysis, performed during a screening phase following the acquisitions, these being carried out in a research context (for study purposes) and not integrated into an operational process;
data capture centered on one type of sensor and not covering a larger spectrum of physiological data, making it more difficult to correlate the data and limiting the robustness of the evaluation and also the false alarm level;
capture of measurements without an integrated synchronization process in the acquisition, making use and analysis more difficult;
no consideration of the environment and of contextualization of the measured physiological information, entailing an increased risk of misinterpretation or misunderstanding of the situation, the capture and the processing being centered only on the physiological aspects of the crew.

This new need makes it necessary to master the measurement, monitoring, processing and rendering (to a third party, to a system) of the state and the behavior of the crew, in a real-time or quasi-real-time context. The measurement has to be taken into account in a process that will respond to this classified state and behavior and will influence the environment of the mission, whether this be real (effective and operational) or simulated (education and training).

The difficulty consists in designing a method and a system that are able to measure and process data characteristic of an operational environment and data from a crew in this environment for contextualization purposes. The method and the system should be able to provide rendering of the results of the processing operations in a short cycle in real time or quasi-real time, either in open circuit mode, called “open loop”, to a third party observer, such as an instructor, for educational and training purposes, or in closed circuit mode, called “closed loop”, to a management system for managing the platform piloted by the crew, for the purpose of helping to accomplish the mission on the basis of the situation of both the crew and its environment.

The expected solution should be able to work both in a real operational context, i.e. on board a real piloted platform, or in a simulated operational context, i.e. on board a simulation of the piloted platform. The solution should also be able to be modular and scalable so as to take into account, on the one hand, technological developments—sensors, processing operations—to be integrated as and when necessary in order to provide better accuracy (precision, segmentation) and better robustness of the results, and take into account, on the other hand, the tailoring to the field and to the operational aim to be covered (choice of components and processing operations).

The main known approaches are work conducted in the automotive field on monitoring systems, studying fatigue and drowsiness of the driver and the position of the vehicle on the traffic lane.

The reference document WP9 “Crew Monitoring” from the European project 7^{th} PCRD ACROSS combines various aspects of crew monitoring in the following versions:
WP9.2 “Crew status characterization for Crew Monitoring system” ACROSS-WP9-DAV-TECH-DEL-0002-D9.2.1;
WP9.3 “Specifications for operational Crew Monitoring System” ACROSS-WP9-DAV-TECH-DEL-0003-D9.3.1;
WP9.3 “Specifications for a Demonstration Crew Monitoring System” ACROSS-WP9-DAV-TECH-DEL-0003-D9.3.2;
WP9.5 “Synthesis of evaluation/demonstration results” ACROSS-WP9-DAV-TECH-DEL-0006-D9.5-2;
WP9.5 “Recommendations for architecture and technologies” ACROSS-WP9-DAV-TECH-DEL-0005-D9.5-3.

The drawbacks of existing solutions include the following:
they do not take into account environmental data (non-physiological data), thereby preventing these measurements from being contextualized and thus limiting the possibilities of detecting inconsistencies, eliminating state and behavior misinterpretations and specifying them, which would make it possible to improve the false alarm rate and the robustness of the obtained results;
they do not generalize, systematize and concentrate the acquisition of data from both sensors and piloted and guided platforms through a standard acquisition bus offering connection/disconnection and synchronization services, which would make it possible to connect any system supplying information and thus to pool this usable information;
they do not provide a framework that makes it possible to combine and chain the rendering production and analysis processing operations (monitoring and analysis view) with real-time or quasi-real-time use of the data, which allows the observers or the system to respond instantly to the state and to the interpreted behavior.

The present invention proposes to address the abovementioned needs and to overcome the described drawbacks.

### SUMMARY OF THE INVENTION

One aim of the present invention is to propose a solution for analyzing the behavior of a subject (team member or crew) that offers:
a multi-sensor approach for simultaneously capturing multiple physiological variables for the purpose of strengthening robustness and reducing the false alarm rate;
taking into account of environmental data (from the piloted-guided platform) in the same way as physiological data and parameters from sensors associated with the subject (or with the crew);
means for synchronizing all of the data acquisition parameters regardless of their origin (sensor, piloted/guided platform, subjective evaluation) or their nature (parameter, videos, audio, etc.) in order to be able to correlate and superimpose them for monitoring, analysis and processing purposes;
a framework that makes it possible to integrate, link and compose analysis and rendering processing models tailored to need and to the desired use;
real-time or quasi-real-time operation of the various components for acquiring, monitoring, processing, recording and rendering data.

Another aim of the present invention is to propose a solution that makes it possible to:
capture and measure the state and the behavior of crew members, through various sensors, by managing the problem of interfacing various sensors, synchronizing and dating the acquired data, recording them and the possibility of replaying them in a synchronized manner;
analyze the state and the behavior of crew members in order to detect and determine physiological and cognitive features such as incapacity, workload, situational awareness, level of learning, etc. by managing the fundamental constraint of real-time or quasi-real-time operation, allowing the system to be inserted into a response loop interfacing with the crew, with observers of the crew, or even with a system managing the environment in which the crew moves. In addition, the solution addresses the problem of tailoring the models to various possible uses, of being able to compose the models and fuse their results, of being able to contextualize (take into account the environment) the information in order to improve the robustness and accuracy of the obtained results.
monitor and render the measured data and the analysis results by managing the problem of providing, in real time or quasi-real time, information relevant to the success of the mission, be this operational (information to operators, to operational systems) or for education and training (information to instructors, trainers, etc.).

The invention will advantageously be applied in fields where it is necessary to track or monitor a crew, an operating team of a platform (aeronautical, land-based, rail, energy, etc.), both in a real operational context (in operation) and in a simulated operational context (education or training).

The preferred applications concern studying and monitoring a crew (“Crew Monitoring”) of a platform (vehicle, system, etc.) both in a simulated context (simulator) and in a real context (for example real aircraft in flight). These applications allow use in order to:
select crews, as it is a means for evaluating the ability of crew members to carry out a mission on the basis of the proposed operational conditions. The selection may relate to an initial selection of helicopter pilots, selection for improvement of helicopter pilots.
educate and train crews, as it is a means for evaluating the performance of a crew, its level of learning and its mastery of the mission, and also its potential for improvement. It may involve educating and training helicopter pilots on a simulator, educating and training on-board control system operators, and educating and training air traffic control operators.
assist in the design of cockpits (aircraft), drivers' cabs (vehicles), or control/monitoring stations, as it is a means for helping to evaluate the effectiveness of a design of a cockpit, driver's cab or control/monitoring station on the basis of the mission, the conditions of the mission and the ability of the crew members. It may involve assisting in the design of cockpits or avionics for helicopters, assisting in the design of cockpits or avionics for airliners and business aircraft.
assist in the evaluation and the certification of new cockpits, drivers' cabs or control/monitoring stations or new on-board electronic equipment. It may involve assisting in the design of control stations for drones (UAV and UCAV), assisting in the design of air traffic control stations.
monitor a crew during operation: makes it possible to tailor the system of the platform (vehicle, plane, helicopter, etc.) on the basis of the state of the crew members and of the operational and environmental situation (reconfiguration of information and of the presentation thereof), alerts and initiation of safety procedures on the basis of the state of the crew members and the operational and environmental situation). This may involve monitoring and assisting pilots of weapons aircraft, monitoring and assisting helicopter pilots, monitoring and assisting airliner pilots and business aircraft pilots during flights, monitoring and assisting operators of drones (UAV and UCAV), monitoring and assisting air traffic controllers.
assist in the customization of entertainment programs and communications services on board airliners.

Advantageously, the use of the proposed solution thus potentially concerns a large number of practical applications that affect various industrial and operational parties.

To achieve these aims, the invention generally consists in setting up a real-time or quasi-real-time platform for acquiring, monitoring, recording, processing and rendering the state and the behavior of a crew. The platform:
is connected to a plurality and diversity of sensors measuring the physiological parameters of the crew;
is connected to the system piloted by the crew (aeronautical or land platform, real or simulated, etc.), which provides parameters about its state, about the environment and about the actions carried out by the crew;
integrates processing and analysis modules at the interface of the captured parameters (from the sensors or the piloted system), but also able to interface for the purpose of composing processing operations;
uses a standardized data bus (DDS) that ensures very easy distribution of processing operations over one or more machines connected to one and the same local area network, and “hot” connection and disconnection of sensors and models, thereby facilitating the stopping and restarting of applications without stopping the measurement session.

The data processed by the platform comprise parameters from physical sensors, parameters from the piloted platform, environmental parameters, crew actions on the piloted platform, scene videos, videos of piloting or guidance equipment and interfaces of the piloted platform, audio communication between crew members and with external operators (for example air traffic controllers), or even with the piloted platform, sounds in the cabin, and notes, markers, annotations or evaluations entered via human-machine interfaces by operators during capture or subsequent analysis.

All of this data is managed in real time or quasi-real time, and the data are dated and synchronized when they are captured. They are archived and stored in real time or quasi-real time. They may be selected to be monitored during acquisition and thus check the correct operation of the acquisition.

They may also be marked or “tagged” by positioning dated markers supporting information of interest. It is possible to operate either in “monitoring” mode, with real-time or quasi-real-time monitoring of the evolution of parameters with listening and viewing of audio and videos, or in “screening” mode, with replaying of the data and display thereof.

It is also possible to select the processing operations that are actually carried out both in “monitoring” mode and in “screening” mode.

This solution makes it possible to provide a capture, processing and rendering environment in which all the information is dated and synchronized, thus making it possible to:
provide real-time or quasi-real-time monitoring of the observable captured data (parameters, videos, audio);
provide real-time or quasi-real-time rendering of the results of the processing operations of detecting, identifying and classifying crew states and behaviors;
contextualize the physiological parameters on the basis of the data from the piloted platform, the actions performed by the crew and the environment, performed by the processing models;
tailor the system to the sensors (in terms of type and in terms of number) required for the envisaged use;
tailor the system to the processing operations required to analyze and develop the required states and behaviors and to render them;
tailor the system to the rendering requirement (content and form) associated with the use made thereof.

To achieve these aims, one subject of the invention is a device for analyzing and monitoring the behavior of a subject moving in an operational environment during a mission, the device comprising:
means for the synchronized acquisition of a plurality of raw data relating to the subject, to the operational environment and to the mission;
means for the on-the-fly processing of the acquired raw data; and
means for generating processed data from the on-the-fly processing operations, providing real-time information about the state and the behavior of the subject;
the device being characterized in that all or some of said means may be implemented so as to activate various operating modes of analyzing and monitoring the behavior of the subject.

According to some alternative or combined embodiments:
the means for synchronized acquisition comprise sensors able to acquire data relating to:
   physiological parameters of the subject;
   technical parameters contextualizing the mission;
   subjective parameters provided by the subject himself or by observers;
   audio and video data relating to the actions of the subject, his movements, and his interactions with the operational environment.
the means for processing the raw data comprise algorithmic analysis models operating in real time.
the means for generating processed data comprise means for providing specialized indicators, notably curves, and alerts expressing situations detected during the mission.
the device additionally comprises means for the deferred processing of the raw data and the processed data.
the device additionally comprises human-machine interfaces able to display instantaneous information about the state and the behavior of the subject.
the various operating modes comprise notably modes of using the evaluation of the state and the behavior of the subject in real time or in deferred mode, of replaying, of rendering and of screening.

The invention also covers a simulator comprising a device for analyzing and monitoring the behavior of a subject as claimed.

The invention extends to an aircraft simulation platform comprising a device for analyzing and monitoring the behavior of a subject as claimed.

Another subject of the invention is a method for analyzing and monitoring the behavior of a subject moving in an operational environment during a mission. The method comprises the following steps:
synchronized acquisition of a plurality of raw data relating to the subject, to the operational environment and to the mission;
on-the-fly processing of the acquired raw data; and
generating processed data from the on-the-fly processing operations, providing real-time information about the state and the behavior of the subject;
said steps making it possible to activate various operating modes of analyzing and monitoring the behavior of the subject.

In alternative or combined embodiments of the method:
the plurality of acquired raw data comprises:
   physiological parameters of the subject;
   technical parameters contextualizing the mission;
   subjective parameters provided by the subject himself or by observers;
   audio and video data relating to the actions of the subject, his movements, and his interactions with the operational environment.
      the step of generating processed data consists in instantaneously providing:
specialized indicators, notably curves;
alerts expressing situations detected during the mission.
   the method additionally comprises a step of deferred processing of the raw data and the processed data, said deferred processing making it possible to generate:
indicators that are presented temporally, in terms of their duration, or their dated punctuality;
any discrepancies or deviations from monitoring in accordance with the procedures that should normally have been followed;
dated markers corresponding notably to detected and declared events;
geographic data positioned on a cartographic view, notably a trajectory;
a set of specialized windows presenting synthetic information, notably gaze tracking, areas looked at by the subject and statistics;
a set of videos and audio selected from the recordings made during the course of the mission.

In another aspect, the invention covers a computer program product comprising non-transitory code instructions for performing the steps of the method as claimed when said program is executed on a computer.

### DESCRIPTION OF THE FIGURES

Various aspects and advantages of the invention will appear in support of the description of one preferred, but nonlimiting, mode of implementation of the invention, with reference to the figures below:

FIG. 1 schematically illustrates the device of the invention in one embodiment;

FIGS. 2a to 2f schematically illustrate various operating modes of the device of the invention;

FIG. 3 schematically illustrates one example of integrating sensors onto a subject;

FIG. 4 schematically illustrates one example of integrating sensors in the environment of a subject;

FIGS. 5a and 5b illustrate examples of screen views of a monitoring and tracking station in the operational phase in real time, according to one embodiment of the invention;

FIG. 6 illustrates three techniques for selecting a time in a replay phase, according to one embodiment of the invention; and

FIG. 7 illustrates one example of a view displayed on the screen of a monitoring and tracking station in a debriefing phase according to one embodiment of the invention.

### DETAILED DESCRIPTION OF THE INVENTION

In general, the device of the invention addresses the problem of measuring and evaluating the state and the behavior of a subject (team member/crew) during a mission that he is conducting, immersed in his operational environment (a platform, a mission system), and of providing the information necessary to act on the system or platform operated by the subject and to act on the subject himself.

The proposed device makes it possible to analyze the behavior of a subject (team member/crew) during a mission, in real or rendered (simulation) conditions, and is based on:
(1) Synchronized acquisition of a plurality of complementary raw parameters/data during a mission. In one embodiment, four types of data or parameters are acquired:
   physiological parameters measuring the personal characteristics of the subject through sensors;
   technical parameters from the platform (for example simulator) providing contextualization elements through state and situation data in the mission (for example flight phase);
   subjective parameters acquired by the subject himself (self-evaluation) or by expert observers (subjective evaluation) in the form of markers, annotation or performance evaluation, providing the operational vision and “field reality” observed by an expert;
   audio and video data recording, illustrating and displaying the actions of the subject, his movements, and interactions with his operational environment.
(2) On-the-fly processing of simultaneously acquired raw data, for the purpose of instantaneously providing a view of the state and the behavior of the subject in the form of a:
   production of rendered (and displayed) processed information in the form of specialized indicators (curves, sampling, etc.);
   production of alerts applied to the processed information expressing a noteworthy situation (subject+operation) encountered during the execution of the mission (crossing of a defined threshold following excessive focus on an instrument), to be brought immediately to the knowledge of the observers.
(3) Deferred processing of the raw data and the data processed during the mission, for the purpose of detecting and rendering the behavioral elements that will be used for the analysis and the pedagogical procedure (debriefing), in the form of:
   indicators (raw or processed parameters, alerts, etc.) presented temporally, in terms of their duration, or their dated punctuality;
   any discrepancies or deviations from monitoring in accordance with the procedures that should normally have been followed;
   dated markers corresponding to detected (for example alerts) and declared events, etc.;
   geographic data positioned on a cartographic view (for example trajectory of the platform);
   a set of specialized windows presenting synthetic information (for example gaze tracking, areas looked at by the subject, statistics, etc.);
   a set of videos and audio selected from the recordings made during the course of the mission.

In the remainder of the description, the expression “real time” is indicated and covers the implementation of the functions in real time or quasi-real time.

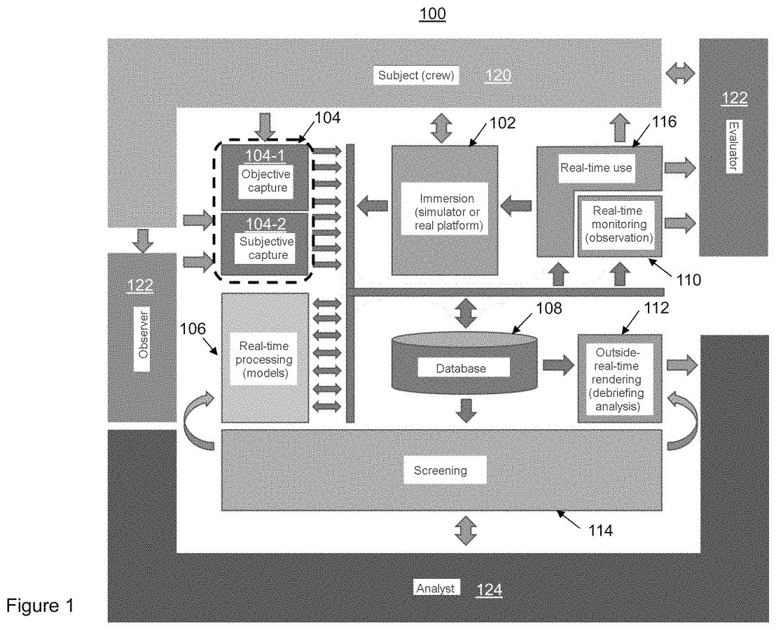

The device of the invention comprises multiple functions and associated means, described with reference to FIG. 1. As illustrated in FIG. 1, the crew monitoring device (100) comprises the following functional modules:

(102) ‘immersion environment’ module: this is the environment close to the subject and the system to which he is physically linked (system or the platform with which the subject interacts). It may be a simulated platform (in the case of a simulator), a real platform (in the case of an aircraft or a workstation, etc.). The immersion environment in the sense of the present invention comprises the integration of the measurement means (interface, arrangement, interference, etc.) into the platform under consideration and the interface with the real or simulated platform in order to extract therefrom state parameters and the actions of the subject.

(104) capture module: this is the set of means/functions (104-1) for “objectively” measuring the various personal parameters of the subject, based on physiological sensors, but also the means/functions (104-2) for “subjectively” measuring (through self-evaluation, evaluation by a third party, event marking) elements of the state and behavior of the subject. These means take into account commercially available sensors known as “COTS” (Commercial Off-The-Shelf) sensors, and capture devices made up of various elementary sensors. Without limitation, the sensors may be video sensors (for example cameras), audio sensors (for example microphones), cardiac sensors (HR/RRI—Heart Rate/RR Interval signals), electrocardiogram (ECG) sensors, skin temperature (Skin T°) sensors, NIRS (near infrared spectroscopy) sensors, electroencephalogram (EEG) sensors, electrooculography (EOG) sensors, electrodermal activity (EDA) sensors, as well as oculometry and gaze direction sensors and inertial units (IMU). The capture function is able to manage the problem of the operational compatibility of the various capture means.

(106) processing and analysis module: this is the set of processing and analysis means/functions implemented in real time, making it possible, on the one hand, to extract information (through filtering, correlation, etc.) from the measurements carried out by the capture means, and, on the other hand, to evaluate the states and behaviors of the subject on the basis of the measured elements. The processing and analysis function is able to fuse the data with the contextualization. The processing operations are carried out through algorithmic analysis models operating in real time.

(108) acquisition and recording module: this is a device interfaced, on the one hand, with the capture module (104) and, on the other hand, with the ‘immersion environment’ module (102) in order to acquire, in real time, all of the parameters and data in a synchronized manner, and while recording them for rendering or screening. The acquisition and recording function is able to implement both communication between all of the real-time components and the recording and archiving of the recorded data.

(110) monitoring module: this is the set of means/functions for monitoring and tracking, in real time, both the parameters measured and acquired on the subject and on the platform (or system) and the results of the processing operations, all with coincidence and in a synchronized manner. The monitoring function applies both to physiological and operational aspects and to technical aspects. The monitoring module may be connected to the usage module (116) in order to take into account the results within the framework of the implementation of the mission conducted by the subject.

(112) rendering module: this is the set of means/functions for providing, outside real time (that is to say in phases following recording but integrated into the mission process, such as for example debriefing), an assessment and a representation of the elements measured (the recorded data and parameters) through an analysis, replay, annotation, and evaluation regarding the state and the behavior of the subject during his mission. The rendering function may be implemented in isolation from the acquisition and recording platform by directly using archived data or in a manner connected to the acquisition and recording platform in replay mode, the latter way also making it possible to implement the functionalities of the monitoring module (110).

(114) screening module: this is the set of means/functions for the deep analysis, outside real time (that is to say in phases following recording and disconnected from the mission), of recorded data for study purposes. This function, in an improvement and maturing process, may be used to evaluate existing analysis models, improve analysis models (configuration, calibration, etc.), and search for new models (deep learning, etc.).

The evolutions of models may be reinjected into the processing module (106) or the rendering module (112) in order to improve their operation and their relevance.

(116) usage module: this is the set of means/functions for reinjecting the results of the real-time analysis of the state and the behavior of the subject both into the management of the system or the operated real or simulated platform (for example through a platform management system) in order to act thereon so as to take into account the state and the behavior of the subject and perform the actions necessary to implement the mission and on the subject himself (for example in the form of an alert, instruction, etc.) so that he is able to best act to implement his mission. The operating function may interact with the monitoring function (110) so as to provide monitoring and a representation of the state and the behavior of the subject. Within the framework of education and training, this use targeting the subject may be carried out by the instructor.

In one embodiment, the data transfers and exchanges of the various modules take place via the DDS (“Data Distribution Service”) communication standard, which is a sophisticated data exchange technology, via a synchronized data bus allowing “hot plug-and-play”.

In some embodiments, depending on the context of the application, some of the functions are automated, such as capture, (real-time) processing, acquisition and recording and monitoring, while others, such as rendering, screening and usage may involve a human processing operation in order to analyze and use the information. Specifically, in the concept of “Crew Monitoring” focusing on the observation and evaluation of the “human”, it is essential to identify the various functions or roles played by the various “human” parties involved. The main (human) roles are defined below:

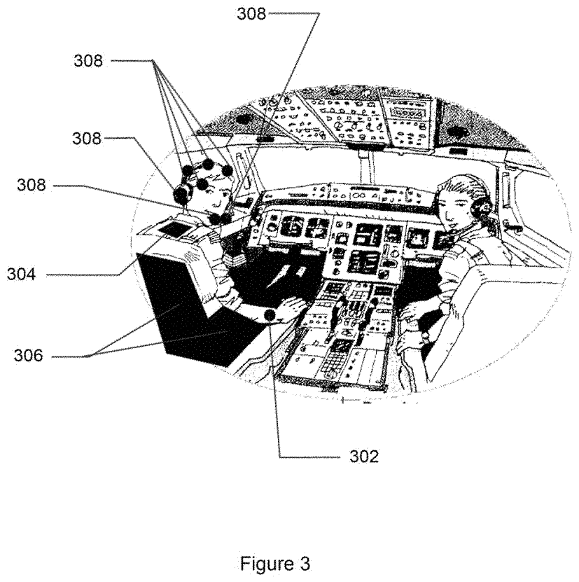

(120) Subject: this is the one or more humans being observed and evaluated during the execution of a mission (real or simulated, in operation or in education/training mode). He may be alone (a pilot) or represent a group of “humans” operating together (for example a crew or a team). The subject is linked directly to his immersion environment (102), such as the cockpit for example, and he wears (carries) the physiological measurement means (104-1). The integration of the monitoring solution into a system (aircraft cockpit for example) should take into account the acceptability to the subject of the presence and the positioning of the sensors with which he interacts and the other equipment in the cockpit. A large number of sensors, to be effective, should be positioned directly on the body or in contact with the body or very close to the body of the subject. FIG. 3 illustrates one example of integrating sensors, which consists in rearranging usual objects already present in the cockpit or the pilot's clothing in order to equip them with sensors so as not to add disruptive elements and thus reduce discomfort. It is thus possible to implement sensors on:
(302) a bracelet in contact with the wrist and the arm of the pilot. The bracelet may be equipped with heart rate, humidity (perspiration), temperature, accelerometer and inertial (IMU) sensors;
(304) a garment in contact with the skin of the pilot. The garment may carry, in a manner integrated into the fabric, body temperature, electrocardiogram ECG and heart rate and acceleration (IMU) sensors;
(306) a seat in contact with the buttocks and the back of the pilot. The seat may be equipped with pressure and temperature sensors;
(308) an (audio) headset in contact with the skull of the pilot. In addition to the microphone, which is a voice sensor, the headset may carry EEG (or even EOG), accelerometer and IMU sensors, and a facial camera.

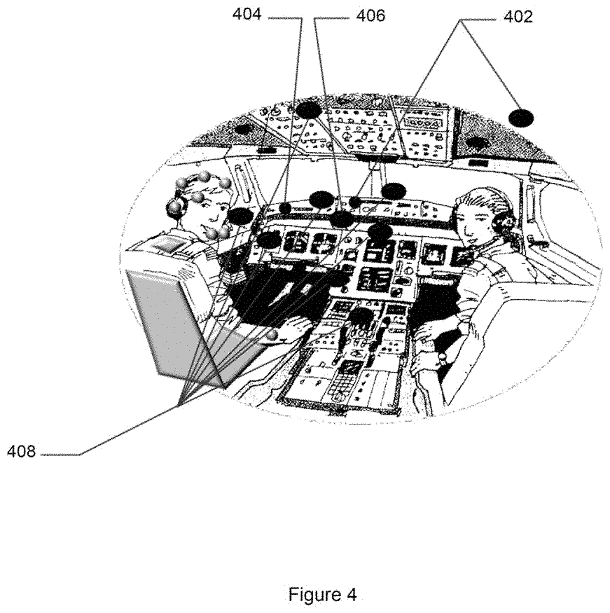

FIG. 4 illustrates one example of integrating remote sensors close to the subject or the crew. The remote sensors comprise various cameras (scene cameras (402), facial cameras (404), 3D cameras (406), gaze tracking cameras (408)), fixed (integrated into the instrument panel and/or the uprights) in the cockpit. These cameras are able to capture the scene (attitude of the pilot, actions performed, etc.), to deduce therefrom postures, facial expressions and any information useful for evaluating behavior.

Returning to FIG. 1, the other parties in the device of the invention are:

(122) Observer: this is the one or more humans monitoring and observing the mission in progress. He observes both the state and the behavior of the subject and the situation of the mission. He may be called upon to comment on and declare his perception of the situation of the subject and the progress of the mission. In some cases, he may also have to interact with the subject (for example the case of an instructor). The observer may be a person or a group of people. An observer may also be an ‘Evaluator’ operating based on the real-time use of the various information about the progress of the mission and the states and behaviors of the subject and the platform. He provides an evaluation of the situation, which may, in some cases, allow him to intervene directly on the subject (for example in the case of an instructor). The evaluator may be a person or a group of people.

(124) Analyst: this is the one or more humans acting at the end of a mission or a set of missions. He performs work on analyzing, screening and formatting of all the recorded data. This work may, as the case may be, be intended to evaluate the subject, or even the platform (in terms of its interface with the subject), but also serve to improve knowledge of human factors and lead to improvements to the analysis models (real time or outside real time). The analyst may be a person or a group of people.

Depending on the implementation contexts, it is possible for several of these roles, in the case of one application, to be grouped together on one and the same person (for example the observer and the evaluator may represent two roles held by an instructor in an education or training application).

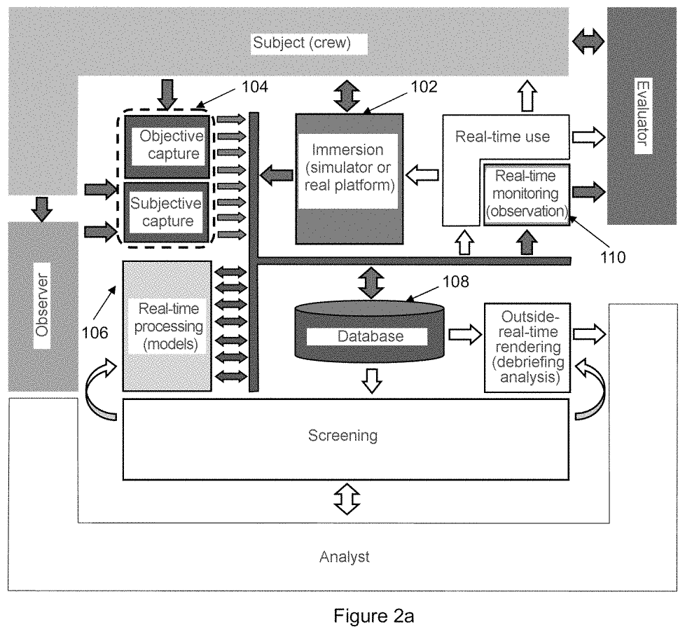

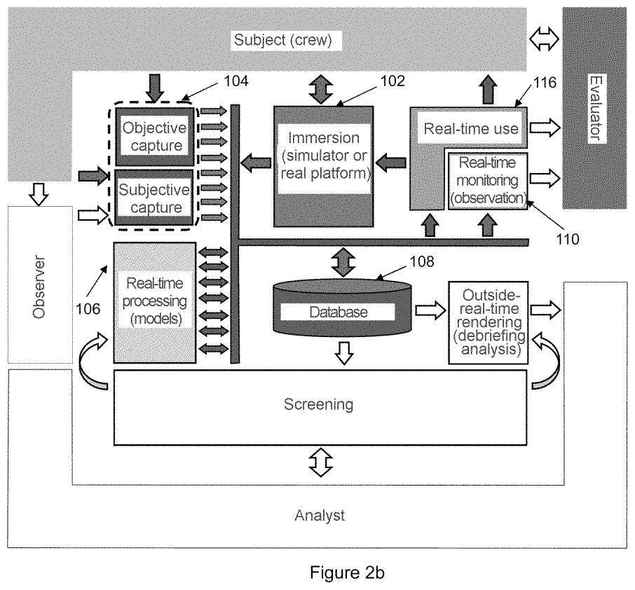

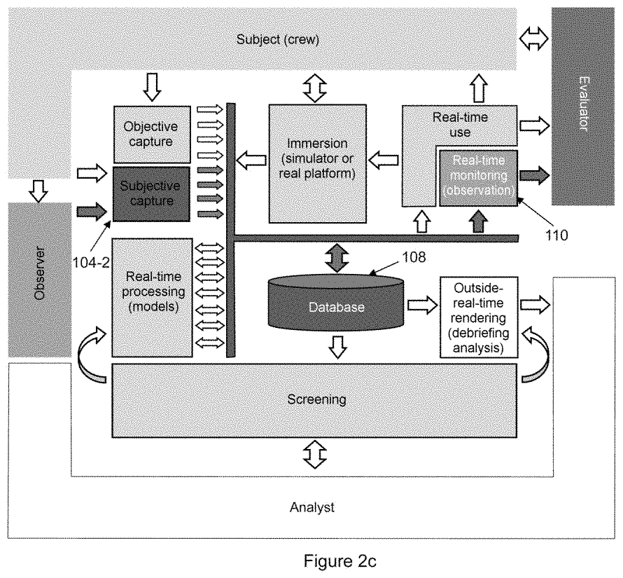

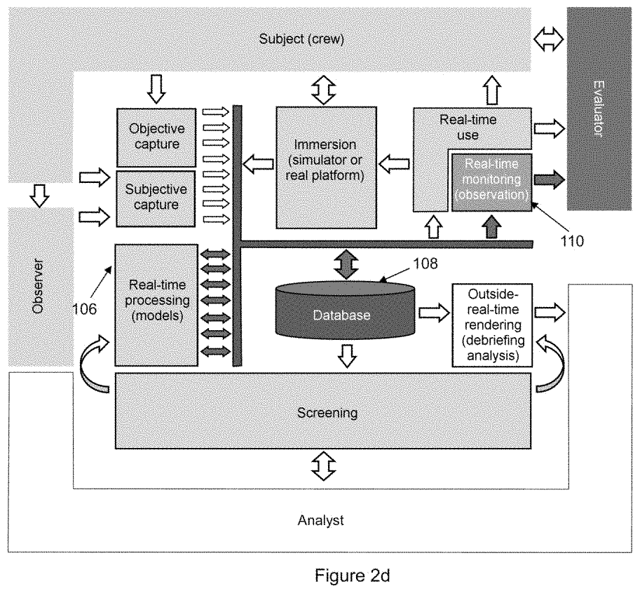

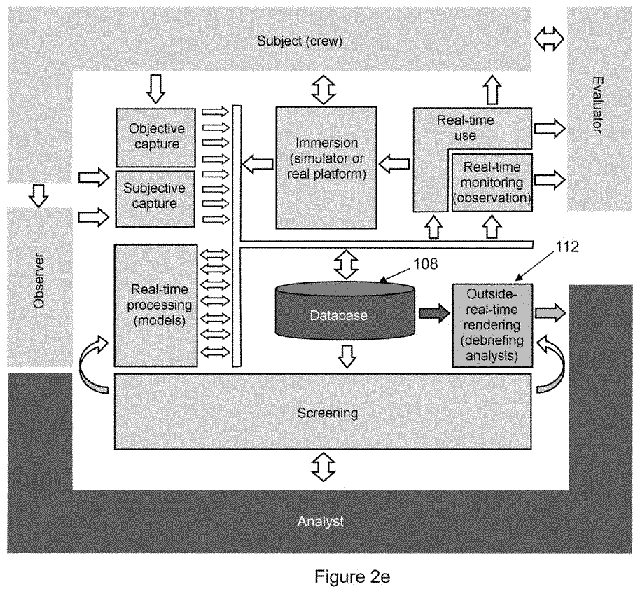

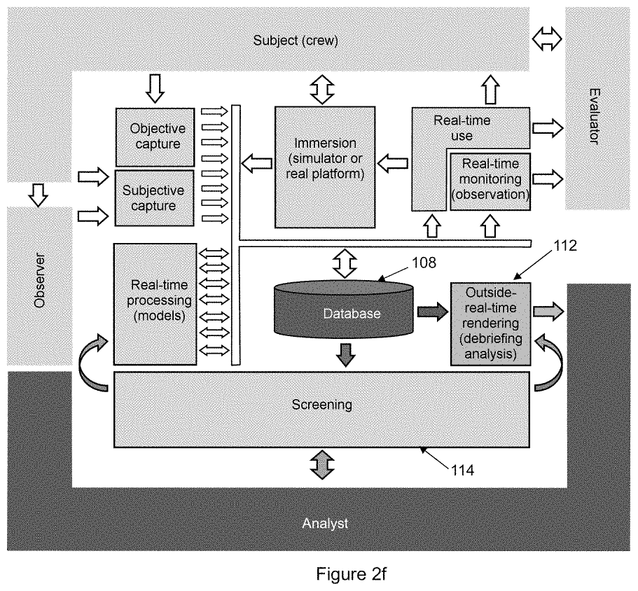

Still depending on the contexts and/or the phases of an implementation, all or some of the modules may be used. Various operating modes are described with reference to FIGS. 2a to 2f, in which the various modules of the platform that are activated are shown in a manner more contrasted than the non-activated modules.

FIG. 2a illustrates what is called the open-loop operating mode. This is a mode that makes it possible to capture, observe and evaluate the state and the behavior of the subject. In this mode, the ‘immersion environment’ (102), capture (104), processing and analysis (106), acquisition and recording (108) and monitoring (110) modules are implemented. The subject (120), the observer (122) and the evaluator (122) are participants. It should be noted that this mode is recommended for monitoring a subject in the education or training phase (presence of an evaluator—subject relationship), as well as for monitoring the performance and efficiency of the human-machine interface of the platform.

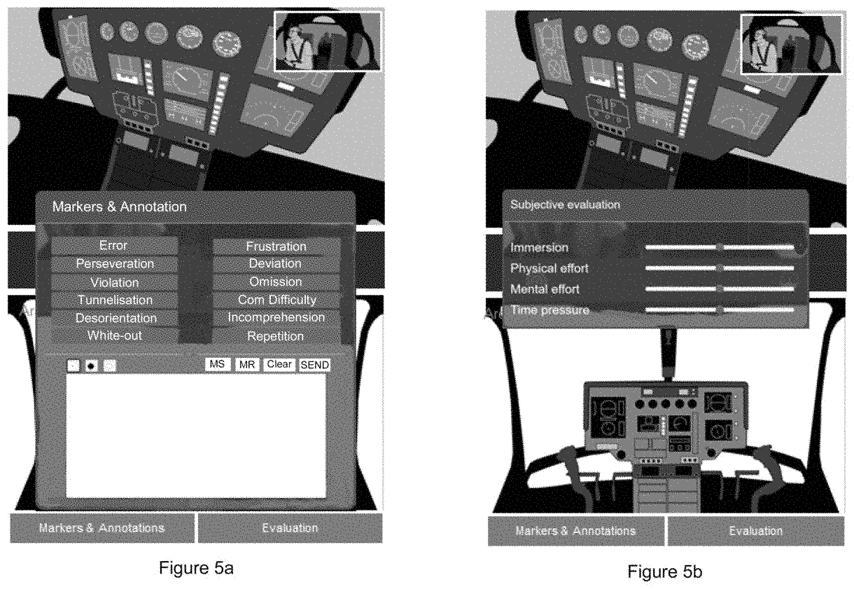

FIGS. 5a and 5b illustrate examples of screen views of a monitoring and tracking station in the operational phase in real time, according to one embodiment of the invention. The views are those displayed on the monitoring and tracking station during the execution of a mission. The view in FIG. 5a shows:
the video channel selected from the various available video channels displayed in thumbnail form and able to be selected;
the representation of the cockpit with display of the point being looked at, and the trace of the gaze over the last few seconds, and also the identification of the element of the cockpit being looked at;
the state of the alerts at the present time (with change of color for example when the alert is activated);
the version of the pilot identification parameters;
the dated evaluation pose from a list of evaluable variables;
the placement of a dated marker from a list of events;
the placement of a dated free annotation using a stylus (free writing).

The view of FIG. 5b adopts the same display except for the markers and annotations, which are replaced by a subjective evaluation input area.

FIG. 2b illustrates what is called the closed-loop operating mode. This is a mode that makes it possible, in real time, to use the evaluation of the state and the behavior of a subject during a mission and to provide a loop back to the system (for example an aircraft) in which he is moving. In this mode, the ‘immersion environment’ (102), capture (104), processing and analysis (106), acquisition and recording (108), monitoring (110) and usage (116) modules are implemented. This mode works without any human intervention (observer, evaluator) other than the subject being studied. It should be noted that this mode is recommended as an advanced human-machine interface means.

FIG. 2c illustrates what is called the replay+declaration operating mode. This is a mode that makes it possible, in real time (i.e. under real conditions), to replay an already recorded session and to be able to add thereto annotations and event marking. In this mode, the subjective capture (104-2), acquisition and recording (108) and monitoring (110) modules are implemented. The observer/evaluator (122) is a participant in this replay+declaration mode. It should be noted that this mode is recommended for the session “debriefing” phases.

FIG. 2d illustrates what is called the replay+processing/analysis operating mode. This is a mode that makes it possible, in real time (i.e. under real conditions), to replay an already recorded session and to be able to add thereto real-time analysis processing operations. In this mode, the processing and analysis (106), acquisition and recording (108) and monitoring (110) modules are implemented. The evaluator (122) is a participant in this replay+processing/analysis mode. It should be noted that this mode is recommended for fine-tuning the processing and analysis models, but also for supplementing and refining the analysis processing operations by performing new analyses.

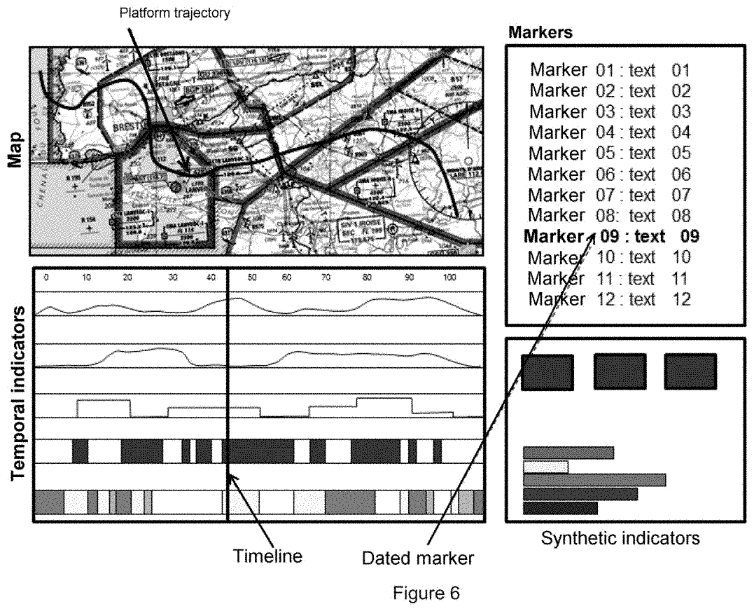

FIG. 2e illustrates what is called the rendering operating mode. This is a mode that makes it possible to analyze the data recorded during a session and to apply thereto outside-real-time (or deferred) processing operations in order to produce and present the resulting information in relation to the application. In this mode, the acquisition and recording (108) and rendering (112) modules are implemented. The analyst (124) is a participant in this rendering mode. It should be noted that this mode is recommended for the debriefing and post-action analysis phases to prepare reports. Access to the recorded information, the rendering thereof and positioning thereof at a given time is achieved through the selection of a time and of the synchronized replay of all of the information over the desired period of time (sequence). The selected time is designated using three complementary techniques, illustrated in FIG. 6:
based on the timeline;
based on a dated marker;
based on a point of the trajectory of the platform.

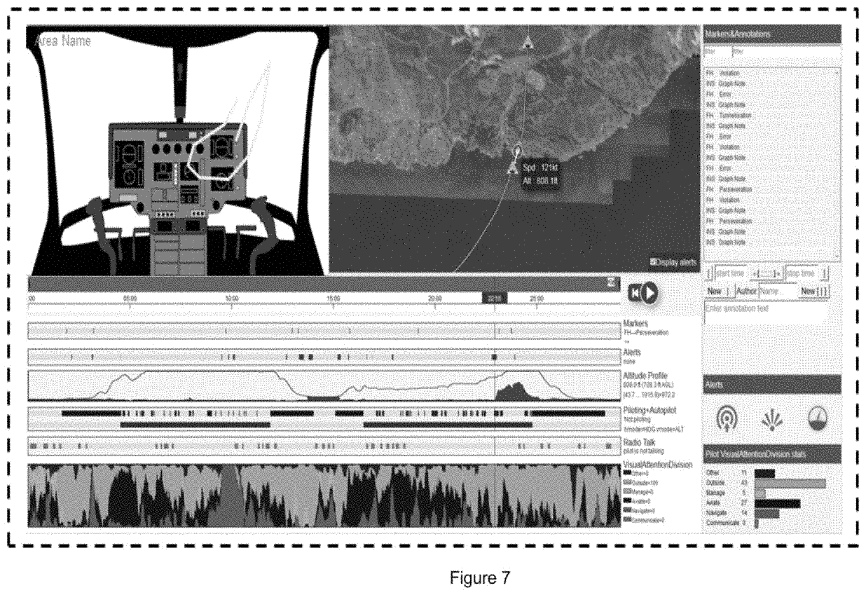

FIG. 7 illustrates one example of a view displayed on the screen of a monitoring and tracking station in a debriefing phase according to one embodiment of the invention. The view displays:
the representation of the cockpit with display of the point being looked at, and the trace of the gaze over the last few seconds, and also the identification of the element of the cockpit being looked at;
the map of the flight area with the trace of the trajectory of the aircraft and the instantaneous position of the aircraft, classified by its velocity and its altitude, enriched with geolocated markers on the trajectory;
a time scale graduated over the entire duration of the exercise, also comprising a timeline indicating the observed time;
the positioning, on the time scale, of the various markers placed, with the identification of the label of the marker located on the timeline;
the positioning, on the time scale, of the various alerts detected, with the elongation corresponding to their activity;
the curve of the flight profile (altitude) over the duration of the exercise, enriched with the curve of the altitude on the ground;
the positioning, on the time scale, of the activated (automatic) piloting modes, with the elongation corresponding to their activity;
the positioning, on the time scale, of the pilot's interventions on the flight controls, with the elongation corresponding to their activity;
the positioning, on the time scale, of the pilot's audio communications, with the elongation corresponding to their activity;
the positioning, on the time scale, of the distribution of the areas looked at by the pilot, segmented by major categories, averaged over the last few seconds, with the display of the percentage of this distribution at the time of the timeline;
the distribution of the areas looked at by the pilot, segmented by major categories, averaged over the selected time window, with the display of the percentage of this distribution;
the status of the alerts (represented in the form of a significant icon) at the time of the timeline;
the list of the various (temporal) markers and annotations classified in chronological order, displayed with a label, which also makes it possible to filter their display;
the free annotation input bar, making it possible to select the time window of its application, its name and the associated text;
the coordinates of the displayed exercise recording, associated with a means for selecting the recording from the list of available recordings.

FIG. 2f illustrates what is called the screening operating mode. This is a mode that makes it possible to carry out heavy processing operations on the data, by exporting the data recorded in screening environments while providing a rendering means that will make it possible to guide these processing operations. In this mode, the acquisition and recording (108), rendering (112) and screening (114) modules are implemented. It should be noted that this mode is recommended for longitudinal monitoring processing operations, for the tailoring and emergence of real-time or outside-real-time analysis models.

By virtue of its modular, integrated and open aspect, the proposed solution provides an open architecture for capturing (measuring), observing, analyzing, evaluating and recording states and contextualized human behavior. It solves the stated problem by way of:
identifying and segmenting a system for measuring and evaluating states and behaviors of a crew into a set of main functions, which lays the foundations for an open modular architecture that is adaptive to the various conceivable application requirements;
a mechanism and an open protocol that make it possible to connect sensors and the piloted-guided platform, processing models and rendering components, and also to synchronize the data acquired and produced (by the processing operations) and record them. The mechanism is based on standards and offers an openness and an adaptability of the system, making it possible to connect new components (be these capture, processing or rendering components) and/or to replace existing components;
taking into account various sensors and also taking into account contextual data provided by the piloted-guided platform, thus improving the quality of the behavior evaluation (better robustness and fewer false alarms);
the ability to take into account, in the processing operations, data previously recorded and processed with a view to longitudinal subject monitoring, making it possible to compare the states and behaviors of one and the same subject at various periods and in repeated or different situations and thus to trace changes in his behavior and his cognitive capacities (example of a learning curve);
the ability to perform capture, processing operations and rendering in real time (that is to say in a short time allowing an immediate action or reaction on the subject, the piloted-guided platform and its environment), making it possible to integrate this measurement and evaluation function into an operational loop (to the subject directly or via an operator, or to the platform directly or via an operator).

To sum up, the present invention provides notable innovations in several aspects:
the application of real time to the measurement of the state and the behavior of the crew and its use;
the ability to perform multi-sensor physiological measurements, and the possibility of expanding the number and the diversity of the sensors;
the ability to synchronously replay already acquired data, but also to enrich them with new annotations and markings, and to add processing operations;
the ability to connect and stack analysis processing operations operating in real time or in deferred time;
the ability to contextualize captured physiological data, using environmental information and parameters captured in real time from the real or simulated platform;
the use of a standardized, synchronized communication bus, interfacing the sensors, the platform, the modules, the rendering, and authorizing “hot plug-and-play”;
the ability to apply longitudinal monitoring to subjects, over definable periods or occurrences.

The present description illustrates one embodiment of the invention, but is not limiting. The example has been chosen so as to allow a good understanding of the principles of the invention, and one specific application, but is not exhaustive, but rather the description should allow a person skilled in the art to provide modifications and implementation variants while keeping the same principles. In particular, adjustments within the scope of those skilled in the art will have to be considered for each application in which a member of personnel interacts with a system and for which his state and his behavior has an influence on the result and the accomplishment of the mission that has been allocated to him. A few cases of customized use of the present invention are thus as follows:
piloting or driving a (real or simulated) mobile platform, such as an aircraft (plane, helicopter, convertible, drone), an item of railway equipment, a vehicle, etc.;
piloting or managing an industrial system, such as a power plant;
piloting or managing a control center such as an air traffic control center, a railway control center, a traffic control center;
monitoring of teams training “live” (in the field) for both civilian and military activities.

1. A device for analyzing and monitoring the behavior of a subject moving in an operational environment during a mission, the device comprising:
means for the synchronized acquisition of a plurality of raw data relating to the subject, to the operational environment and to the mission;
means for the on-the-fly processing of the acquired raw data; and
means for generating processed data from the on-the-fly processing operations, providing real-time information about the state and the behavior of the subject;
the device being characterized in that all or some of said means may be implemented so as to activate various operating modes of analyzing and monitoring the behavior of the subject.

2. The device as claimed in claim 1, wherein the synchronized acquisition means comprise sensors able to acquire data relating to:
physiological parameters of the subject;
technical parameters contextualizing the mission;
subjective parameters provided by the subject himself or by observers;
audio and video data relating to the actions of the subject, his movements, and his interactions with the operational environment.

3. The device as claimed in claim 1, wherein the means for the on-the-fly processing of the raw data comprise means for filtering and correlating the acquired raw data and means for analyzing the data based on algorithmic models operating in real time.

4. The device as claimed in claim 1, wherein the means for generating processed data comprise means for providing specialized indicators, notably curves, and alerts expressing situations detected during the mission.

5. The device as claimed in claim 1, additionally comprising means for the deferred processing of the raw data and the processed data.

6. The device as claimed in claim 1, additionally comprising human-machine interfaces able to display instantaneous information about the state and the behavior of the subject.

7. The device as claimed in claim 1, wherein the various operating modes comprise notably modes of using the evaluation of the state and the behavior of the subject in real time or in deferred mode, of replaying, of rendering and of screening.

8. A simulator comprising a device for analyzing and monitoring the behavior of a subject as claimed in claim 1.

9. An aircraft simulation platform comprising a device for analyzing and monitoring the behavior of a subject as claimed in claim 1.

10. A method for analyzing and monitoring the behavior of a subject moving in an operational environment during a mission, the method comprising the following steps:
synchronized acquisition of a plurality of raw data relating to the subject, to the operational environment and to the mission;
on-the-fly processing of the acquired raw data; and
generating processed data from the on-the-fly processing operations, providing real-time information about the state and the behavior of the subject;
said steps making it possible to activate various operating modes of analyzing and monitoring the behavior of the subject.

11. The method as claimed in claim 10, wherein the plurality of acquired raw data comprises:
physiological parameters of the subject;
technical parameters contextualizing the mission;
subjective parameters provided by the subject himself or by observers;
audio and video data relating to the actions of the subject, his movements, and his interactions with the operational environment.

12. The method as claimed in claim 10, wherein the step of generating processed data consists in instantaneously providing:
specialized indicators, notably curves;
alerts expressing situations detected during the mission.

13. The method as claimed in claim 10, additionally comprising a step of deferred processing of the raw data and the processed data, said deferred processing making it possible to generate:
indicators that are presented temporally, in terms of their duration, or their dated punctuality;
any discrepancies or deviations from monitoring in accordance with the procedures that should normally have been followed;
dated markers corresponding notably to detected and declared events;
geographic data positioned on a cartographic view, notably a trajectory;
a set of specialized windows presenting synthetic information, notably gaze tracking, areas looked at by the subject and statistics;
a set of videos and audio selected from the recordings made during the course of the mission.

14. A computer program product comprising non-transitory code instructions for performing the steps of the method as claimed in claim 10 when said program is executed on a computer.
